(19) United States
(12) Patent Application Publication
(10) Pub. No.: US 2017/0231705 A1
(43) Pub. Date: Aug. 17, 2017

(54) MULTI-LAYER MEDICAL GLOVE

(71) Applicant: PAUL MADISON, BOCA RATON, FL (US)

(72) Inventor: PAUL MADISON, BOCA RATON, FL (US)

(73) Assignee: Double Glove LLC, BOCARATON, FL (US)

(21) Appl. No.: 15/433,160

(22) Filed: Feb. 15, 2017

Related U.S. Application Data
(60) Provisional application No. 62/295,617, filed on Feb. 16, 2016.

(51) Int. Cl. A61B 42/10; A41D 31/00; A41D 19/00

(52) U.S. Cl. CPC A61B 42/10; A41D 19/0006; A41D 31/0083; A41D 19/0082; A41D 2400/34

(57) ABSTRACT

A multi-layer glove has a first glove layer and a second glove layer. A pathogen resistant substance is positioned between the first glove layer and the second glove layer.

### RELATED APPLICATIONS

The present patent application claims the benefit of U.S. Provisional Application No. 62/295,617, filed Feb. 16, 2016, entitled “THE DOUBLE GLOVE”, in the name of the same inventor and which is incorporated herein by reference in its entirety.

### TECHNICAL FIELD

The present invention relates generally to the field of medical devices, and more specifically, to a multi-layer glove system for preventing infection and the transmission of pathogens wherein the multi-layer glove system may be comprised of two layers that are wet when placed on a user's hand. Antibiotics may be placed in between the two layers to help prevent any infection when using needles or ripping of a layer.

### BACKGROUND

During medical procedures, medical and dental personnel may come in contact with a number of potential harmful substances. These substances may pose a potential health risk to these medical personnel. Because of this, medical personnel generally wear personal protective equipment (PPE) such as gloves when performing medical procedures. Medical gloves may be worn to prevent cross-contamination between medical personnel and patients. Medical gloves may protect medical personnel from viruses, toxins, hepatitis, acquired immune deficiency syndrome, and a plethora of other risks face medical personnel every day.

Medical gloves may be made from different polymers including latex, nitrile rubber, polyvinyl chloride and neoprene. Medical gloves may come unpowdered or powdered with cornstarch to lubricate the gloves, making them easier to put on the hands. Medical gloves are generally impervious to the solutions and solvents carrying these dangers pathogens as well as other risks medical personnel face every day.

However, most medical gloves are thin and may easily be cut or pricked by needles or scalpels. Any opening in the medical glove may allow blood or other fluid to be transferred from a patient to the medical personnel. This may allow the possibility of pathogens being transferred to medical personnel.

Due to the dangers of medical gloves being torn/rip during a medical procedure, medical glove manufacturers have attempted to provide a more durable medical glove. Unfortunately, more durable medical gloves tend to lose dexterity.

Therefore, it would be desirable to provide a system and method that overcomes the above.

### SUMMARY

In accordance with one embodiment, a multi-layer glove is disclosed. The multi-layer glove has a first glove layer and a second glove layer. A pathogen resistant substance is positioned between the first glove layer and the second glove layer.

In accordance with one embodiment, a multi-layer glove is disclosed. The multi-layer glove has a first glove layer having an open end and a plurality of plurality of separate sheaths for each finger and a thumb of a user. A second glove layer is positioned over the first glove layer and has an open end and a plurality of plurality of separate sheaths for each finger and the thumb of the user. A pathogen resistant substance is positioned between the first glove layer and the second glove layer. A seal is formed between the first glove layer and the second glove layer. The seal is formed around a perimeter of the open end of the first glove layer and the open end of the second glove layer.

In accordance with one embodiment, a multi-layer glove is disclosed. The multi-layer glove has a first glove layer having an open end and a plurality of plurality of separate sheaths for each finger and a thumb of a user. The first glove layer is formed of a stretchable air and liquid impermeable material. A second glove layer is positioned over the first glove layer and has an open end and a plurality of plurality of separate sheaths for each finger and the thumb of the user. The second glove layer is formed of the stretchable air and liquid impermeable material. A pathogen resistant substance is positioned between the first glove layer and the second glove layer. A seal is formed between the first glove layer and the second glove layer. The seal is formed around a perimeter of the open end of the first glove layer and the open end of the second glove layer.

### BRIEF DESCRIPTION OF THE DRAWINGS

The present application is further detailed with respect to the following drawings. These figures are not intended to limit the scope of the present invention but rather illustrate certain attributes thereof.

FIG. 1 is a prospective view of a medical glove according to one aspect of the present application;

FIG. 2 is a side view of the medical glove of FIG. 1 in accordance with one aspect of the present application; and

FIG. 3 is a cross-sectional view of the medical glove depicted in FIG. 1 according to one aspect of the present application.

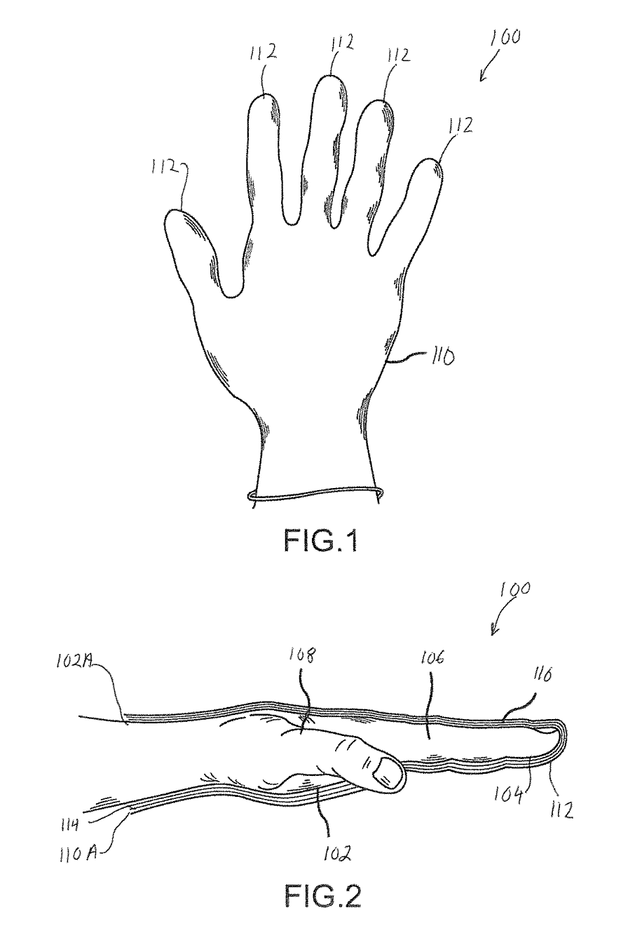

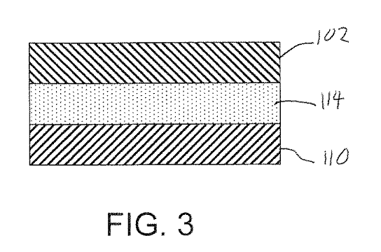

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary system and method relates to a multi-layer medical glove system for preventing infection and the transmission of pathogens. The system may be comprised of two layers. Antibiotics may be placed in between the two layers to help prevent any infection when using needles or ripping of a layer.

Referring now to the FIGs., a medical glove 100 may be seen. The medical glove 100 may be formed of a first glove layer 102. The first glove layer 102 may be formed out of a stretchable air and liquid impermeable material. For example, different polymers such as latex, nitrite rubber, polyvinyl chloride and neoprene may be used. The above is given as examples and should not be seen in a limiting manner.

The first glove layer 102 may have a plurality of separate sheaths 104 for each finger 106 and the thumb 108 of the user. An opening 102A may be formed on one end of the first glove layer 102. The opening 102A may allow a user to insert his/her hand into the medical glove 100.

The medical glove 100 may have a second glove layer 110. The second glove layer 102 may be positioned over the first glove layer 102. The second glove layer 110 may be formed out of a stretchable air and liquid impermeable material. For example, different polymers such as latex, nitrile rubber, polyvinyl chloride and neoprene may be used to form the second glove layer 110. The above is given as examples and should not be seen in a limiting manner.

The second glove layer 110 may have a plurality of separate sheaths 112 for each finger 106 and the thumb 108 of the user. The separate sheaths 112 of the second glove 110 may align with corresponding sheaths 104 of the first glove 102. An opening 112A may be formed on one end of the second glove layer 110. The opening 110A may allow a user to insert his/her hand into the medical glove 100.

A solution 114 may be positioned between the first glove 102 and the second glove 110. The solution 114 may be a pathogen resistant solution. The solution 114 may prevent the spread of pathogens and thereby prevent infections between the first glove 102 and the second glove 110 if either one or both of the gloves 102 and/or 110 are punctured or ruptured. The solution 114 may be a thin film of antiseptic, antibiotic, antibacterial, alcohol, iodine, or sterilant solution.

A seal 116 may be formed between the first glove 102 and the second glove 110. The seal 116 may be formed to secure the perimeter of the opening 102A of the first glove layer 102 to the perimeter of the opening 1 10A of the second glove layer 110. The seal 116 may be used to prevent the solution 114 from leaking out between the first glove layer 102 to the perimeter of the opening 110A of the second glove layer 110.

While embodiments of the disclosure have been described in terms of various specific embodiments, those skilled in the art will recognize that the embodiments of the disclosure may be practiced with modifications within the spirit and scope of the claims.

1. A multi-layer glove comprising:
a first glove layer;
a second glove layer; and
a pathogen resistant substance positioned between the first glove layer and the second glove layer.

2. The multi-layer glove of claim 1, wherein the first glove layer is formed of a stretchable air and liquid impermeable material.

3. The multi-layer glove of claim 1, wherein the second glove layer is formed of a stretchable air and liquid impermeable material.

4. The multi-layer glove of claim 1, comprising a seal formed between the first glove layer and the second glove layer.

5. The multi-layer glove of claim 1, comprising a seal formed between the first glove layer and the second glove layer, the seal formed around a perimeter of an opening of the first glove layer and an opening of the second glove layer.

6. The multi-layer glove of claim 1, wherein the first glove layer is formed from one of: latex, nitrile rubber, polyvinyl chloride, neoprene and similar polymers.

7. The multi-layer glove of claim 1, wherein the first glove layer is formed from one of: latex, nitrile rubber, polyvinyl chloride, neoprene and similar polymers.

8. The multi-layer glove of claim 1, wherein the pathogen resistant substance is one of: an antiseptic, antibiotic, antibacterial, alcohol, iodine, and similar sterilant solution.

9. The multi-layer glove of claim 1, wherein the first glove layer has an open end and a plurality of plurality of separate sheaths for each finger and a thumb of a user.

10. The multi-layer glove of claim 1, wherein the second glove layer has an open end and a plurality of plurality of separate sheaths for each finger and a thumb of a user.

11. A multi-layer glove comprising:
a first glove layer having an open end and a plurality of plurality of separate sheaths for each finger and a thumb of a user;
a second glove layer positioned over the first glove layer and having an open end and a plurality of plurality of separate sheaths for each finger and the thumb of the user;
a pathogen resistant substance positioned between the first glove layer and the second glove layer; and
a seal formed between the first glove layer and the second glove layer, the seal formed around a perimeter of the open end of the first glove layer and the open end of the second glove layer.

12. The multi-layer glove of claim 11, wherein the first glove layer is formed of a stretchable air and liquid impermeable material.

13. The multi-layer glove of claim 11, wherein the second glove layer is formed of a stretchable air and liquid impermeable material.

14. The multi-layer glove of claim 11, wherein the first glove layer is formed from one of: latex, nitrile rubber, polyvinyl chloride, neoprene and similar polymers.

15. The multi-layer glove of claim 11, wherein the first glove layer is formed from one of: latex, nitrile rubber, polyvinyl chloride, neoprene and similar polymers.

16. The multi-layer glove of claim 11, wherein the pathogen resistant substance is one of: an antiseptic, antibiotic, antibacterial, alcohol, iodine, and similar sterilant solution.

17. A multi-layer glove comprising:
a first glove layer having an open end and a plurality of plurality of separate sheaths for each finger and a thumb of a user, wherein the first glove layer is formed of a stretchable air and liquid impermeable material;
a second glove layer is positioned over the first glove layer and has an open end and a plurality of plurality of separate sheaths for each finger and the thumb of the user, wherein the second glove layer is formed of the stretchable air and liquid impermeable material;
a pathogen resistant substance positioned between the first glove layer and the second glove layer; and
a seal formed between the first glove layer and the second glove layer, the seal formed around a perimeter of the open end of the first glove layer and the open end of the second glove layer.

18. The multi-layer glove of claim 17, wherein the first glove layer is formed from one of: latex, nitrile rubber, polyvinyl chloride, neoprene and similar polymers.

19. The multi-layer glove of claim 17, wherein the second glove layer is formed from one of: latex, nitrile rubber, polyvinyl chloride, neoprene and similar polymers.

20. The multi-layer glove of claim 17, wherein the pathogen resistant substance is one of: an antiseptic, antibiotic, antibacterial, alcohol, iodine, and similar sterilant solution.
